(19) United States
(12) Patent Application Publication
(10) Pub. No.: US 2005/0239117 A1
(43) Pub. Date: Oct. 27, 2005

(54) Biochip measuring method and biochip measuring apparatus

(76) Inventors: Takeo Tanaami, Tokyo (JP); Yumiko Sugiyama, Tokyo (JP); Yasunori Suzuki, Tokyo (JP)

Correspondence Address: SUGHRUE MION, PLLC, 2100 PENNSYLVANIA AVENUE, N.W., SUITE 800, WASHINGTON, DC, 20037, US

(21) Appl. No.: 11/104,172

(22) Filed: Apr. 11, 2005

(30) Foreign Application Priority Data
Apr. 21, 2004 (JP) P.2004-124897

(51) Int. Cl. C12Q 1/68; G06F 19/00; G01N 33/48; G01N 33/50; G06K 9/00

(52) U.S. Cl. 435/6; 702/20; 382/128

(57) ABSTRACT

A biochip measuring method of measuring data of a biochip having a plurality of sites, having the steps of obtaining a plurality of captured images of a biochip, whose brightness are different respectively, and which are captured with different measurement setting, and combining images of each site or numerical value of each site based on brightness of each site in each captured image, to produce one synthetic data corresponding to the biochip.

### CROSS-REFERENCE TO RELATED APPLICATIONS

This application is based upon and claims the benefit of priority from the prior Japanese Patent Applications No. 2004-124897, filed on Apr. 21, 2004, the entire contents of which are incorporated herein by reference.

### BACKGROUND OF THE INVENTION

1. Field of the Invention

The present invention relates to a biochip measuring method and a biochip measuring apparatus which measure data of a biochip having a plurality of sites, and more particularly to improvements in the biochip measuring method and the biochip measuring apparatus which measure an image for a biochip such as a DNA micro-array in a broad dynamic range over a broad measured area.

2. DESCRIPTION OF THE RELATED ART

The following document is related to an apparatus for measuring a biochip over the broad measured area of the biochip.

JP-A-2001-311690 is referred to as A related art.

FIGS. 5 and 6 are schematic views showing the essence of one example of a biochip measuring apparatus as described in JP-A-2001-311690. FIG. 5 shows a biochip measuring apparatus of scanless type, and FIG. 6 shows a biochip measuring apparatus of scan type in which a DNA chip is scanned in a transverse direction (direction orthogonal to an optical axis).

In FIG. 5, an excited light (e.g., laser beam) emitted from a light source 101 is made parallel by a lens 102, and incident upon a lens L through a micro-lens array MA and an aperture AP to become parallel rays, which are then incident upon a dichroic mirror 103. The micro-lens array MA having a micro-lens ML is employed to increase the brightness, but may not be provided.

The excited light reflected from the dichroic mirror 103 is condensed by an objective lens 106 to illuminate a sample face of a DNA chip 8.

Owing to this illuminating light, a sample emits a fluorescence (having a different wavelength from the excited light). The fluorescence goes back to the objective lens 106 to be incident upon the dichronic mirror 103. The fluorescence from the sample transmitted through the dichroic mirror 103 passes through a filter 5 shielding any other light than fluorescence and enters a lens 108. With this lens 108, a fluorescent image on the sample face of the DNA chip 8 is formed on a photodetector 111. The photodetector may be a camera, for example.

A biochip measuring apparatus of scan type as shown in FIG. 6 has the same constitution in principle as shown in FIG. 5, but is different in that the DNA chip 8 is scanned in a direction (of the arrow MV) perpendicular to the incident direction of the excited light, and a glass or plastic substrate capable of transmitting the fluorescence is employed as the substrate for the DNA chip 8 to dispose the DNA on this substrate (on the lower side in the figure).

Such biochip measuring apparatus of scanless or scan type can read images at plural sites on the DNA chip. When a dark site and a light site are present on the same DNA chip, the power of excited light, the gain of photodetector, or the light integrating time of photodetector is appropriately changed or adjusted to make the image unsaturated and unburied in the noise.

However, the above biochip measuring apparatus has the following problems to be solved.

(1) In the case that a dynamic range of brightness is broad, it is difficult to obtain an image having appropriate brightness that is neither too light nor too dark.

(2) In the case that the power of excited light, the gain of photodetector or the light integrating time of photodetector is changed, it is difficult to obtain a correlation between plural sheets of images. For example, when a photo-multiplier is used as the photodetector, it is required to measure the same sample under different settings, and acquire the relationship between images by making the regression calculation employing the measured data at every time of measurement, because the gain has non-linearity. That is unfavorable in the respects of time and precision. If a lot of data are obtained by changing the settings for one site, it is unclear which data to employ.

(3) Moreover, the image must be digitized, but it takes a lot of time to digitize a plurality of sheets of images. Where the number of sites is M and N sheets of images are provided per site, M×N processings are required.

(4) Since a plurality of sheets of images are necessary to be compared with the eye of a user, it is difficult for the user to grasp or compare as one biochip.

### SUMMARY OF THE INVENTION

The object of the invention is to provide a biochip measuring method and a biochip measuring apparatus which are capable of measuring a biochip having a broad dynamic range of sites correctly in a short time, so that the whole biochip is easily grasped or compared.

The invention provide a biochip measuring method of measuring data of a biochip having a plurality of sites, has the steps of: obtaining a plurality of captured images of a biochip, whose brightness are different respectively, and which are captured with different measurement setting; and combining images of each site or numerical value of each site based on brightness of each site in each captured image, to produce one synthetic data corresponding to the biochip.

Therefore, the biochip having sites of a broad dynamic range is correctly measured in a short time, and the whole biochip is easily grasped.

The biochip measuring method further has the step of: comparing brightness with a saturation level or a noise level for each site between the plurality of captured images.

The biochip measuring method further has the steps of: in a case of comparing brightness with the saturation level for each site, replacing an image or numerical value of a site where brightness thereof reach the saturation level with an image or numerical value of the same site of another captured image having the site whose brightness is less than the saturation level, and in a case of comparing brightness with the noise level for each site, replacing an image or numerical value of a site where brightness thereof is less than or equal to the noise level with an image or numerical value of the same site of another captured image having the site whose brightness is more than the noise level.

In the biochip measuring method, a variance value of brightness is employed for the comparison of brightness and the noise level.

The biochip measuring method further has the step of: editing an image of each site based on brightness of each site, and then converting the image of each site to numerical value, to produce the synthetic data.

The biochip measuring method further has the step of: converting all of the plurality of captured images, and then editing a numerical value of each site based on the numerical value of each site, to produce the data.

The biochip measuring method further has the step of: editing a numerical value of each site, and then editing an image of each site, to reconstruct one image.

In the biochip measuring method, a relationship between the measurement setting and brightness of an image is calibrated before measurement.

In the biochip measuring method, a relationship between the measurement setting and brightness of an image is calibrated based on brightness of a marker substance blended into a sample at a time of measurement.

In the biochip measuring method, the measurement setting is set such that brightness of the plurality of captured images are represented in equal magnification, power of 2, exponential, logarithm, or geometrical progression.

In the biochip measuring method, the plurality of captured images are intermediate synthetic images that are created by making addition and subtraction of the plurality of captured images.

In the biochip measuring method, the measurement setting is varied by changing an illuminating light power, a gain of a photodetector, or the measuring time.

In the biochip measuring method, the biochip is a chip or a micro-array of DNA, RNA, protein, glycolipid, or metabolite.

The invention also provides a biochip measuring apparatus which measuring data of a biochip having a plurality of sites, including: an image processing section which produces a synthetic image with employing the biochip measuring method.

According to the biochip measuring method and the biochip measuring apparatus, the biochip having sites of a broad dynamic range is correctly measured in a short time, and the whole biochip is easily grasped.

### BRIEF DESCRIPTION OF THE DRAWING

FIGS. 1A and 1B are views showing a specific example of an image captured in a short time according to the invention;

FIGS. 2A and 2B are views showing a specific example of an image captured in a long time according to the invention;

FIGS. 3A and 3B are views showing a specific example of a synthetic image created by a method of the invention;

FIG. 4 is a schematic view showing the essence of one example of a biochip measuring apparatus that implements the method of the invention;

FIG. 5 is a schematic view showing the essence of one example of a conventional biochip measuring apparatus of scanless type; and

FIG. 6 is a schematic view showing the essence of one example of a conventional biochip measuring apparatus of scan type.

### DETAILED DESCRIPTION OF THE PREFERRED EMBODIMENTS

The present invention will be described below in detail with reference to the accompanying drawings. This invention is related to a biochip measuring apparatus and a biochip measuring method which measures data of a biochip having a plurality of sites.

An embodiment of the biochip measuring method according to the invention will be described below. In the embodiment, a plurality of captured images of a biochip, whose brightness are different brightness, and which are captured with different measurement setting are selectively used in part based on the brightness value itself of each site in each captured image, and the images of each site or the numerical value of each site are combined, to produce one synthetic data corresponding to the biochip.

A measuring step will be described below.

(1) A plurality of sheets of images are captured by changing the setting (e.g., image capturing time) without moving the mounting position of a DNA chip on the biochip measuring apparatus.

(2) The position of a site in each image is decided, employing the known information regarding the site location (local position) on the DNA chip array.

(3) The following replacement is made while examining the brightness for all the sites in each image.

(3.1) First of all, the lightest captured image, for example, the image captured with employing a CCD camera as the photodetector for an image capturing time of T1 second (e.g., 30 seconds), is prepared.

(3.2) A site with saturated brightness is searched from them, and the image of the site (referred to as image data, or simply data, and so on) is replaced, in a unit of site, with the image captured darker one level, for example, the image at the same site that is captured for an image capturing time of T2 seconds (e.g., 10 seconds). At this time, the information about the replacement site and the image capturing time (T1 and T2) is recorded separately.

(3.3) The site where brightness thereof is saturated is searched again. That is, the site where the brightness is saturated after the above replacement is searched. For the site where the brightness is saturated, the image is replaced, in a unit of site, with the image captured darker further one level, for example, the image at the same site that is captured for an image capturing time of T3 (e.g., one second). At this time, the information about the replacement site and time T1 and T3 is recorded separately.

(3.4) The above steps are repeated till reaching a state where all the sites are unsaturated. The re-measurement is made under the darker conditions, if necessary.

(4) The following step is performed, employing the replaced image and the recorded information.

(4.1) The conversion rate K1 is decided so that the lightest site may not exceed the maximum gradation (65535 in this case) in consideration of a predetermined dynamic range of image (e.g., 16 bit). For example, K1=0.9.

(4.2) The final synthetic image is created by converting the brightness of each site in the replaced image, employing this conversion rate K1 and the time information (T1 to T3) for each site.

For example, the brightness is reduced in the following way.

For the saturated site with the capturing time of T1, the brightness is increased at a magnification of K1×(T3/T1)=0.9/30.

For the saturated site with the capturing time of T2, the brightness is increased at a magnification of K1×(T3/T2)=0.9/10.

For the site with the capturing time of T3, the brightness is increased at a magnification of K1.

Thereby, the darker site where the image is not captured for the capturing time of T3 can be confirmed on the screen of the final synthetic image. This is because the background noise that is significant for T3 is represented smaller at the site for T1. Also, for the site saturated for T1 and not compared, the brightness is easily compared on the screen of final synthetic image. Moreover, since this image is only one sheet, the image analysis for digitization is necessary only once, and the regression calculation for measuring (calibration) is unnecessary.

Thereby, the darker site where the image is not captured for the capturing time of T3 can be confirmed on the screen of the final synthetic image. This is because the background noise that is significant for T3 is represented smaller at the site for T1. Also, for the site saturated for T1 and not compared, the brightness is easily compared on the screen of the final synthetic image.

Since this final synthetic image is only one sheet, the image analysis for digitization is necessary only once. In this case, the regression calculation for measuring (calibration) is unnecessary.

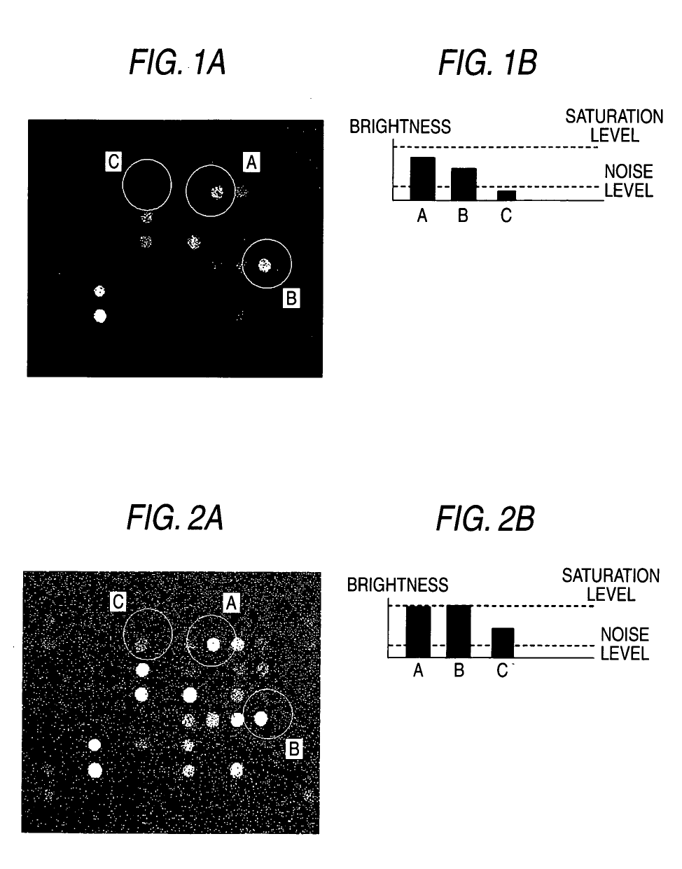

FIGS. 1A to 3B show a specific example of the captured image and the final synthetic image. FIG. 1A shows an image captured for a short time (T1). FIG. 1B shows the brightness level representation for the sites A, B and C encircled in FIG. 1A. The brightness of sites A and B lies between the noise level and the saturation level, whereby the image is observable, and a difference in the brightness between two sites is clear. However, since the brightness of C site is below the noise level, the image is not observable.

FIGS. 2A and 2B show images captured for a long time (T2). Regarding the brightness of the sites A, B and C, the sites A and B are saturated, without difference in the brightness, as shown in FIG. 2B, but the site C is at an intermediate level between the saturated brightness and the noise, whereby the image is observable.

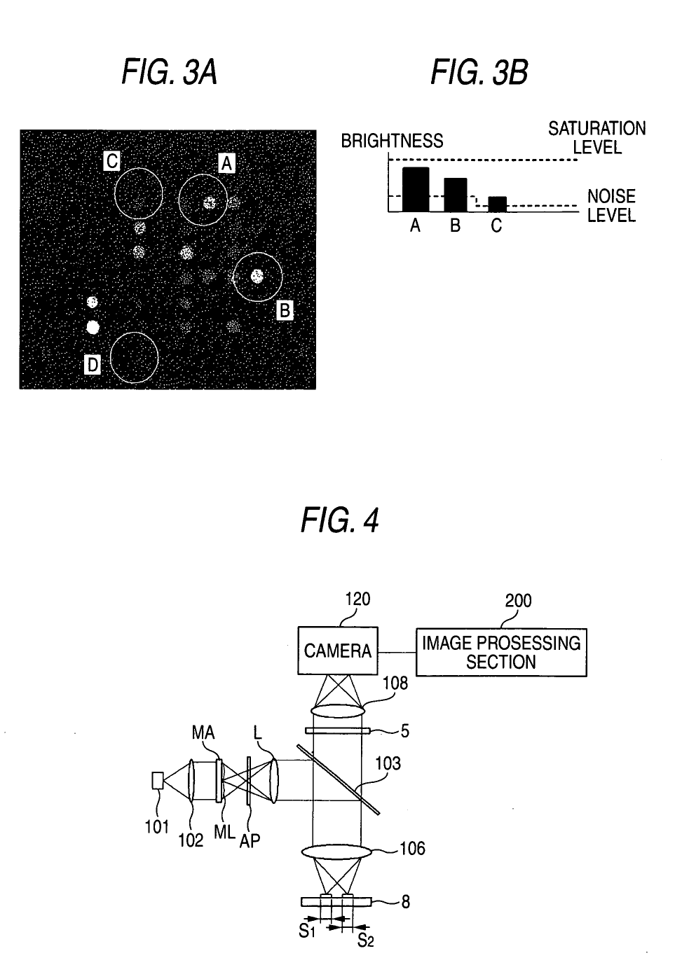

If these two captured images are subjected to image processing by the inventive method, the synthetic image is produced, as shown in FIG. 3A. That is, in the lightest captured image of FIG. 2A, the images at the sites A and B with saturated brightness are replaced with the images at the same sites that are captured darker one level in FIG. 1A. Since other sites in FIG. 2A are not saturated, no replacement is made. Thereafter, the conversion rate K1 is acquired in the above way, and the brightness of replaced image at each site is converged, based on the conversion rate K1 and the time information for each site, to produce the synthetic image of FIG. 3A.

In this case, the noise level is larger at sites A and B and smaller at site C, as shown in FIG. 3B.

For an encircled part D in FIG. 3A to be useful in another embodiment as will be described later, the explanation is omitted.

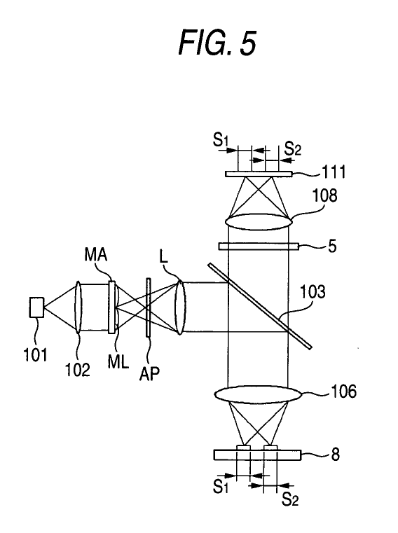

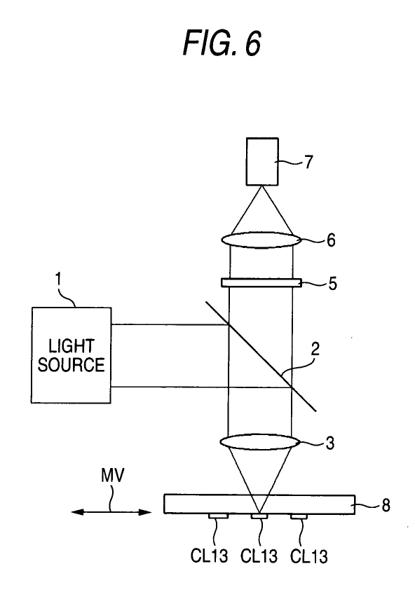

FIG. 4 is a schematic view of the essence showing one example of the biochip measuring apparatus that implements the above biochip measuring method. In the embodiment, the biochip measuring apparatus of scanless type as shown in FIG. 5 for measuring data of a biochip having a plurality of sites is employed. The different points from FIG. 5 are that a camera 120 as a photodetector 111 is used and an image processing section 200 is newly added.

The camera 120 can capture a fluorescent image on the sample face for a set image capturing time. The image processing section 200 has a control section for presetting the image capturing time of the camera 120 and driving the camera to capture the image for the set time, a storage section (not shown) for saving each image captured by the camera 120 while changing the image capturing time in succession, a site position detecting section (not shown) for detecting the position of site in each image based on the known information, and an image processing section (not shown) for performing the processing of producing the synthetic image by the inventive method by reading each saved image.

The operation of the biochip measuring apparatus having this constitution will be described below. The operation in which an excited light from the light source 101 is applied on the sample face, and an image on the sample face is captured by the camera 120 is the same as the reader of FIG. 5, and not described here. The inherent operation of the invention will be only described below.

In capturing the image on the sample face, the position of the sample is fixed. For the set image capturing time, the images on the sample face are captured in succession, and saved in the image processing section 200.

In the image processing section 200, the position of site in each image is detected and decided, employing the known information about the location of site in the biochip.

After capturing, the luminous energies for all the sites in each captured image are examined and a lightest captured image is selected from the captured images. And the site with saturated brightness is searched from the image. If there is any site with saturated brightness, the image of the site is replaced with the image at the same site that is captured darker one level. When replaced, each image capturing time for the image before and after replacement is stored associated with the site in the image processing section 200.

After replacing the image, the image processing section 200 searches the replaced image for the site with saturated brightness again. If there is any site with saturated brightness, the image of the site is replaced with the image at the same site that is captured darker one level. Each image capturing time for the image before and after replacement is stored associated with the site in the image processing section 200.

This replacing step is repeated until the site with saturated brightness disappears.

Then, the image processing section 200 performs the following step, employing the replaced image and the recorded information, to produce the synthetic image.

First of all, the conversion rate K1 is decided so that the lightest site may not exceed the maximum gradation in consideration of a predetermined dynamic range of image.

And the synthetic image is created by converting the brightness of each site in the replaced image, employing this conversion rate and the time information for each site.

The biochip measuring method and the biochip measuring apparatus of the invention are not limited to the above embodiment, but may be modified or changed in various ways without departing from the spirit or scope of the invention. The modifications are listed in the following.

(1) A difference in the brightness between captured images may be made by adjusting not only the image capturing time but also the power of excited light (laser) or the gain of the camera (photodetector). The relationship between the measurement setting of the reader and the brightness (gradation) is decided beforehand.

(2) Replacement of the image may begin with not the lighter captured image (e.g., image with an image capturing time of 30 seconds) as in the embodiment, but the darker captured image (e.g., image with a capturing time of 1 second).

In this case, when no site is seen at the location where it should be, namely, the average value of brightness of the site is almost equivalent to the background (background light) measured at the location where there is no sample, it is considered that the site is buried in the noise, whereby the site is replaced with the site lighter one level. A determination whether or not the site is buried in the noise can be made more correctly by performing the analysis of variance using a dispersion in the standard deviation of the brightness.

For the background light, any other location than the site (D in FIG. 3A), or the site that is known not to connect with the sample (site without spot, or site with spot of gene apparently not existent in the sample) may be employed.

(3) A way of producing the lighter captured image (hereinafter referred to as a light image) and the darker captured image (hereinafter referred to as a dark image) may be made as follows.

(3.1) The numerical values of parameters for changing the light or shade of image (e.g., integration time, laser power) are measured under the same settings multiple times, in which the darkest image employs one sheet of image (e.g., image captured for an image capturing time of one second), and the lighter images employ sequentially the added images (e.g., two sheets of one-second images are added, or ten sheets of one-second images are added).

(3.2) The parameter for changing the brightness is selected under the condition where the brightness is the power of two (e.g., one second, two seconds, four seconds, eight seconds, and sixteen seconds). The images produced in these series employ the bit shift operation in the division to produce the synthetic image, because the brightness is the power of two (e.g., division of 4 is conversion from 16 bit to 4 bit). This operation using the CPU has an advantage that the operation process is fast, and an operation error is unlikely to occur.

(3.3) If the parameter series is logarithmic series, exponential series or geometrical progression, a broader dynamic range is obtained with a smaller number of sheets of images.

(3.4) A determination may be made after creating a new intermediate synthetic image by the addition of a number of images. For example, the lighter sites in a group of images measured according to the power of two or logarithm may employ the addition (addition/subtraction) of darker sites measured previously. If the additive image is not used, the darker captured image is discarded, when the lightest captured image is employed, but the measured data can be utilized effectively and thoroughly by making the addition.

For example, when there are three captured images of 1 second, 10 seconds and 30 seconds, a series of one second, 10 seconds, 11 seconds (=one second+10 seconds), 30 seconds, 31 seconds (=30 seconds+one second), 40 seconds (=30 seconds+10 seconds) and 41 seconds (=30 seconds+11 seconds) are created. Moreover, a subtractive image of 9 seconds (=10 seconds−one second) may be provided.

(4) The operation sequence may be made as follows.

With the above method, a digitization process is made after synthesis of the images. Thereby, the digitization process for each site is greatly reduced below N (sheets)×M (sites) times. However, the image may be re-synthesized by performing the operation in advance, and based on the operation result. In this case, the operation time is required, but there is an advantage that the total image is easily grasped by one sheet of image.

(5) Calibration of reader

With the above method, the relationship between the measurement setting and the brightness of image is calibrated before measurement, but marker molecules may be blended in a measurement sample liquid before measurement, and the measurement setting may be calibrated by the marker molecules at the time of measurement.

1. A biochip measuring method of measuring data of a biochip having a plurality of sites, comprising the steps of:
obtaining a plurality of captured images of a biochip, whose brightness are different respectively, and which are captured with different measurement setting; and
combining images of each site or numerical value of each site based on brightness of each site in each captured image, to produce one synthetic data corresponding to the biochip.

2. The biochip measuring method according to claim 1, further comprising the step of:
comparing brightness with a saturation level or a noise level for each site between the plurality of captured images.

3. The biochip measuring method according to claim 2, further comprising the steps of:
in a case of comparing brightness with the saturation level for each site, replacing an image or numerical value of a site where brightness thereof reach the saturation level with an image or numerical value of the same site of another captured image having the site whose brightness is less than the saturation level, and
in a case of comparing brightness with the noise level for each site, replacing an image or numerical value of a site where brightness thereof is less than or equal to the noise level with an image or numerical value of the same site of another captured image having the site whose brightness is more than the noise level.

4. The biochip measuring method according to claim 3,
wherein a variance value of brightness is employed for the comparison of brightness and the noise level.

5. The biochip measuring method according to claim 1, further comprising the step of:
editing an image of each site based on brightness of each site, and then converting the image of each site to numerical value, to produce the synthetic data.

6. The biochip measuring method according to claim 1, further comprising the step of:
converting all of the plurality of captured images, and then editing a numerical value of each site based on the numerical value of each site, to produce the data.

7. The biochip measuring method according to claim 6, further comprising the step of:
editing a numerical value of each site, and then editing an image of each site, to reconstruct one image.

8. The biochip measuring method according to claim 1,
wherein a relationship between the measurement setting and brightness of an image is calibrated before measurement.

9. The biochip measuring method according to claim 1,
wherein a relationship between the measurement setting and brightness of an image is calibrated based on brightness of a marker substance blended into a sample at a time of measurement.

10. The biochip measuring method according to claim 1,
wherein the measurement setting is set such that brightness of the plurality of captured images are represented in equal magnification, power of 2, exponential, logarithm, or geometrical progression.

11. The biochip measuring method according to claim 1,
wherein the plurality of captured images are intermediate synthetic images that are created by making addition and subtraction of the plurality of captured images.

12. The biochip measuring method according to claim 1, wherein the measurement setting is varied by changing an illuminating light power, a gain of a photodetector, or the measuring time.

13. The biochip measuring method according to claim 1, wherein the biochip is a chip or a micro-array of DNA, RNA, protein, glycolipid, or metabolite.

14. A biochip measuring apparatus which measuring data of a biochip having a plurality of sites, comprising:
an image processing section which produces a synthetic image with employing the biochip measuring method according to claim 1.
